(19) United States
(12) Patent Application Publication
(10) Pub. No.: US 2025/0032121 A1
(43) Pub. Date: Jan. 30, 2025

(54) SYSTEMS AND METHODS FOR TREATING ANEURYSMS

(71) Applicant: Covidien LP, Mansfield, MA (US)

(72) Inventors: Mehdi M. Rashidi, Irvine, CA (US); Robert A. Pecor, Aliso Viejo, CA (US); Junwei Li, Irvine, CA (US); Nelson M. Siu, Encinatas, CA (US); Belinko K. Matsuura, Encinitas, CA (US); David G. Matsuura, Solana Beach, CA (US)

(21) Appl. No.: 18/784,182

(22) Filed: Jul. 25, 2024

Related U.S. Application Data
(60) Provisional application No. 63/516,316, filed on Jul. 28, 2023.

(51) Int. Cl. A61B 17/12; A61B 17/00

(52) U.S. Cl. CPC A61B 17/12113; A61B 17/00234; A61B 17/12031; A61B 17/12172; A61B 2017/00238; A61B 2017/00292; A61B 2017/00477; A61B 2017/00557; A61B 2017/12054

(57) ABSTRACT

Treatment of aneurysms can be improved by delivering an occlusive implant (e.g., an expandable braid) to an aneurysm sac in conjunction with an embolic element (e.g., coils, embolic material). A treatment system for such treatment can include an expandable member that releasably engages the occlusive implant to an engagement portion of a conduit. When the expandable member is collapsed, the conduit is disengaged from a proximal hub of the occlusive implant. The conduit and expandable member may then be retracted while the occlusive implant remains at the treatment site.

### CROSS-REFERENCE TO RELATED APPLICATIONS

This application claims the benefit of and priority to U.S. Provisional Patent Application Ser. No. 63/516,316 filed Jul. 28, 2023, the entire disclosure of which is incorporated by reference herein.

### TECHNICAL FIELD

The present technology relates to systems, devices, and methods for treating intracranial aneurysms.

### BACKGROUND

An intracranial aneurysm is a portion of an intracranial blood vessel that bulges outward from the blood vessel's main channel. This condition often occurs at a portion of a blood vessel that is abnormally weak because of a congenital anomaly, trauma, high blood pressure, or for another reason. Once an intracranial aneurysm forms, there is a significant risk that the aneurysm will eventually rupture and cause a medical emergency with a high risk of mortality due to hemorrhaging. When an unruptured intracranial aneurysm is detected or when a patient survives an initial rupture of an intracranial aneurysm, vascular surgery is often indicated. One conventional type of vascular surgery for treating an intracranial aneurysm includes using a microcatheter to dispose a platinum coil within an interior volume of the aneurysm. Over time, the presence of the coil should induce formation of a thrombus. Ideally, the aneurysm's neck closes at the site of the thrombus and is replaced with new endothelial tissue. Blood then bypasses the aneurysm, thereby reducing the risk of aneurysm rupture (or re-rupture) and associated hemorrhaging. Unfortunately, long-term recanalization (i.e., restoration of blood flow to the interior volume of the aneurysm) after this type of vascular surgery occurs in a number of cases, especially for intracranial aneurysms with relatively wide necks and/or relatively large interior volumes.

Another conventional type of vascular surgery for treating an intracranial aneurysm includes deploying a flow diverter within the associated intracranial blood vessel. The flow diverter is often a mesh tube that causes blood to preferentially flow along a main channel of the blood vessel while blood within the aneurysm stagnates. The stagnant blood within the aneurysm should eventually form a thrombus that leads to closure of the aneurysm's neck and to growth of new endothelial tissue, as with the platinum coil treatment. One significant drawback of flow diverters is that it may take weeks or months to form aneurysmal thrombus and significantly longer for the aneurysm neck to be covered with endothelial cells for full effect. This delay may be unacceptable when risk of aneurysm rupture (or re-rupture) is high. Moreover, flow diverters typically require antiplatelet therapy to prevent a thrombus from forming within the main channel of the blood vessel at the site of the flow diverter. Antiplatelet therapy may be contraindicated shortly after an initial aneurysm rupture has occurred because risk of re-rupture at this time is high and antiplatelet therapy tends to exacerbate intracranial hemorrhaging if re-rupture occurs. For these and other reasons, there is a need for innovation in the treatment of intracranial aneurysms. Given the severity of this condition, innovation in this field has immediate life-saving potential.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A shows a perspective view of a system for treating an aneurysm in accordance with the present technology.

FIG. 1B shows an enlarged view of a distal portion of the treatment system of FIG. 1A in accordance with the present technology.

FIGS. 1C and 1D are sectioned views of occlusive members in an expanded state in accordance with the present technology.

FIG. 2 shows an embolic kit according to the present technology.

FIGS. 3A-3G depict an example method of treating an aneurysm with the treatment system of the present technology.

FIG. 4A shows a side cross-sectional view of a treatment system in accordance with aspects of the present technology.

FIG. 4B is an enlarged detail cross-sectional view of a portion of the treatment system shown in FIG. 4A.

FIGS. 5A and 5B illustrate side cross-sectional views of the treatment system in an engaged and a disengaged state, respectively.

FIGS. 6A and 6B illustrate perspective views of the treatment system in an engaged and a disengaged state, respectively.

### DETAILED DESCRIPTION

Methods for treating intracranial aneurysms in accordance with at least some embodiments of the present technology include positioning an expandable occlusive member within the aneurysm and introducing an embolic element between the occlusive member and an aneurysm wall. Introduction of the embolic element both fills space within the aneurysm cavity and deforms the occlusive member from a first expanded state to a second expanded state to fortify the occlusive member at the neck of the aneurysm. Deformation of the occlusive member from a first expanded state to a second expanded state provides the additional advantage of giving visual confirmation to the physician that the delivered amount of embolic element sufficiently fills the aneurysm cavity. In addition to providing a structural support and anchor for the embolic element, the occlusive member provides a scaffold for tissue remodeling and diverts blood flow from the aneurysm. Moreover, the embolic element exerts a substantially uniform pressure on the occlusive member towards the neck of the aneurysm, thereby pressing the portions of the occlusive member positioned adjacent the neck against the inner surface of the aneurysm wall such that the occlusive member forms a complete and stable seal at the neck.

Once the occlusive member has deployed within the aneurysm and the embolic element has been delivered, the occlusive member may be detached from the delivery assembly. Suitable detachment mechanisms must be as small as possible so as to be guided through the fine bore of the catheter to the treatment site, while on the other hand they must securely and reliably produce detachment of the intrasaccular implant. Absent a reliable detachment of the intrasaccular implant, withdrawal of the delivery conduit and catheter may cause unintended removal of the occlusive member from the cavity to be occluded and thus injure and/or rupture of the wall of the cavity or vessel. In some embodiments, a detachment mechanism as described herein can rely on an expandable member such as a balloon, and may be used to facilitate reliable, controlled detachment of the occlusive member.

The occlusive member can be implanted in body cavities or blood vessels. In addition to the occlusive member, the treatment system can comprise a delivery conduit and a catheter (e.g., an elongated sheath). The occlusive member and the delivery conduit can be coupled together such that both can be slid in the catheter in the longitudinal direction. For example, the occlusive member can be coupled to a distal portion of the delivery conduit, and the delivery conduit can include an engagement portion configured to releasably engage with a proximal hub of the occlusive member. In some implementations, the conduit engagement portion can be configured to be slidably disposed within a proximal hub of the occlusive member, such that the hub circumferentially surrounds the conduit engagement portion. An expandable member (e.g., an inflatable balloon) can be coupled to an outer surface of the conduit engagement portion such that, when the engagement portion is disposed within the proximal hub, the expandable member is positioned radially between a radially outer surface of the conduit and a radially inner surface of the proximal hub.

The expandable member carried by the conduit engagement portion can be configured to transition between an engaged configuration and a disengaged configuration with respect to the proximal hub. In the engaged configuration, the expandable member is radially expanded (e.g., inflated) such that the expandable member exerts a radially outward force on the radially inner surface of the proximal hub. This radially outward force prevents axial movement of the proximal hub with respect to the engagement portion. For example, the radially outer surface of the expandable member can frictionally engage and/or mechanically interlock with the radially inner surface of the proximal hub such that axial movement is prevented. In the disengaged configuration, the expandable member is radially collapsed (e.g., deflated), thereby reducing or eliminating the radially outward force exerted by the expandable member upon the radially inner surface of the proximal hub. By virtue of this reduced or eliminated force, the conduit engagement portion can be slidably retracted with respect to the proximal hub, thereby releasing the occlusive member from the delivery conduit.

In some implementations, the delivery conduit is configured to pass one or more embolic elements therethrough for intrasaccular delivery. In some embodiments, the occlusive member, for instance a self-expandable braided implant, can define an implant lumen in fluid communication with the conduit lumen when in the engaged state. The implant lumen can be configured to pass an embolic element (e.g., embolic material, hydrogel) therethrough to a position distal to at least a portion of a distal face of the occlusive member. The embolic element may be passed through the delivery conduit and delivered to the treatment site (e.g., intrasaccularly at a position distal to the occlusive member). Once the occlusive member and any embolic elements are deployed, the expandable member can be collapsed to disengage the occlusive member from the delivery conduit at the conduit engagement portion. After the delivery conduit has been disengaged, the delivery conduit can be retracted, and the occlusive member may remain in position at the treatment site.

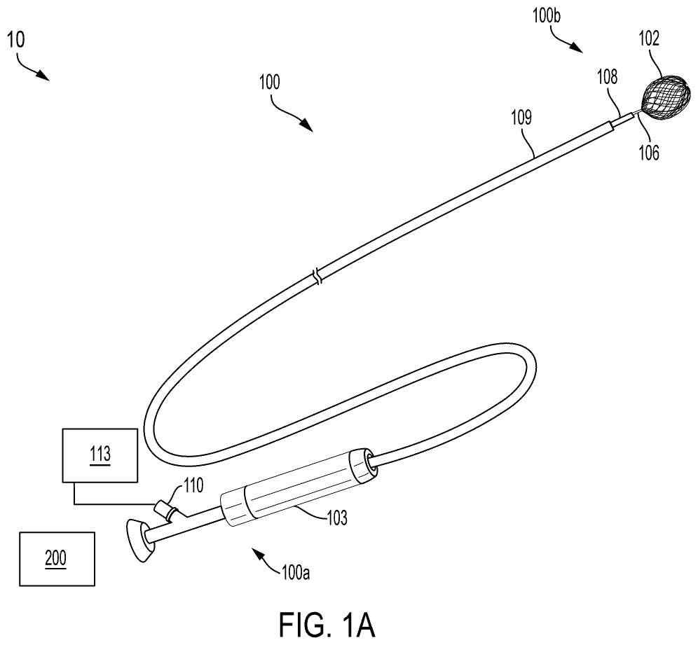

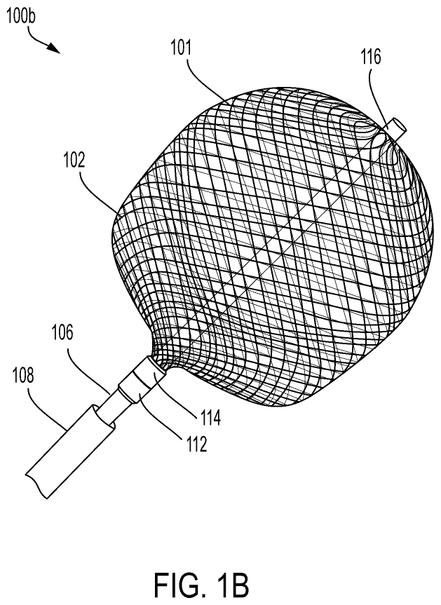

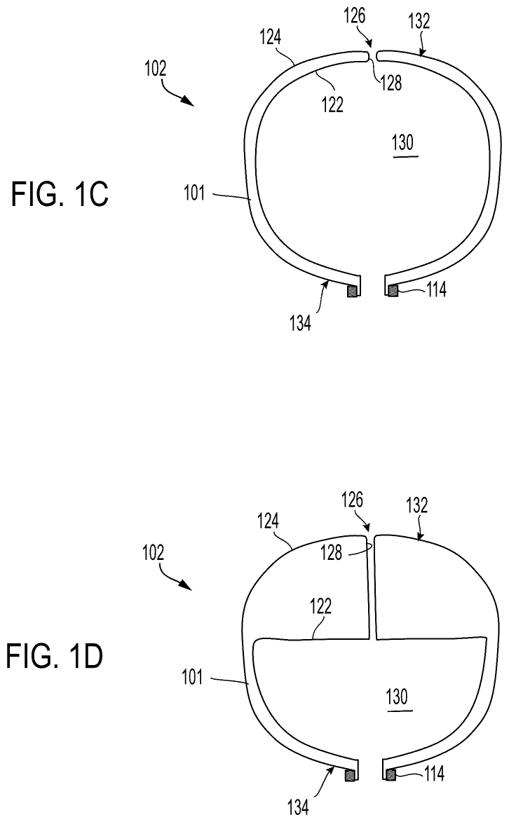

Specific details of systems, devices, and methods for treating intracranial aneurysms in accordance with embodiments of the present technology are described herein with reference to FIGS. 1A-6B. Although these systems, devices, and methods may be described herein primarily or entirely in the context of treating saccular intracranial aneurysms, other contexts are within the scope of the present technology. For example, suitable features of described systems, devices, and methods for treating saccular intracranial aneurysms can be implemented in the context of treating non-saccular intracranial aneurysms, abdominal aortic aneurysms, thoracic aortic aneurysms, renal artery aneurysms, arteriovenous malformations, tumors (e.g. via occlusion of vessel(s) feeding a tumor), perivascular leaks, varicose veins (e.g. via occlusion of one or more truncal veins such as the great saphenous vein), hemorrhoids, and sealing endoleaks adjacent to artificial heart valves, covered stents, and abdominal aortic aneurysm devices among other examples. Furthermore, it should be understood, in general, that other systems, devices, and methods in addition to those disclosed herein are within the scope of the present disclosure. For example, systems, devices, and methods in accordance with embodiments of the present technology can have different and/or additional configurations, components, procedures, etc. than those disclosed herein. Moreover, systems, devices, and methods in accordance with embodiments of the present disclosure can be without one or more of the configurations, components, procedures, etc. disclosed herein without deviating from the present technology.

### I. OVERVIEW OF SYSTEMS OF THE PRESENT TECHNOLOGY

FIG. 1A illustrates a view of a system 10 for treating intracranial aneurysms according to one or more embodiments of the present technology. As shown in FIG. 1A, the system 10 comprises a treatment system 100 and an embolic kit 200 for use with one or more components of the treatment system 100. The treatment system 100 may comprise an occlusive member 102 (shown in an expanded state) detachably coupled to a delivery system, and the delivery system may be configured to intravascularly position the occlusive member 102 within an aneurysm. The embolic kit 200 may comprise one or more substances or devices that alone or in combination form an embolic element that is configured to co-occupy the internal volume of the aneurysm with the occlusive member 102. In some embodiments, the treatment system 100 may be configured to deliver the embolic element (and/or one or more precursors thereof) to the aneurysm. Additionally or alternatively, the system 10 may include a separate delivery system (not shown) for delivering the embolic element (and/or one or more precursors thereof) to the aneurysm cavity.

As shown in FIG. 1A, the treatment system 100 has a proximal portion 100a configured to be extracorporcally positioned during treatment and a distal portion 100b configured to be intravascularly positioned within a blood vessel (such as an intracranial blood vessel) at a treatment site at or proximate an aneurysm. The treatment system 100 may include a handle 103 at the proximal portion 100a, the occlusive member 102 at the distal portion 100b, and a plurality of elongated shafts or members extending between the proximal and distal portions 100a and 100b. In some embodiments, such as that shown in FIG. 1A, the treatment system 100 may include a first elongated shaft 109 (such as a guide catheter or balloon guide catheter), a second elongated shaft 108 (such as a microcatheter) configured to be slidably disposed within a lumen of the first elongated shaft 109, and an elongated member 106 configured to be slidably disposed within a lumen of the second elongated shaft 108. In some embodiments, the treatment system 100 does not include the first elongated shaft 109 and only includes the second elongated shaft 108.

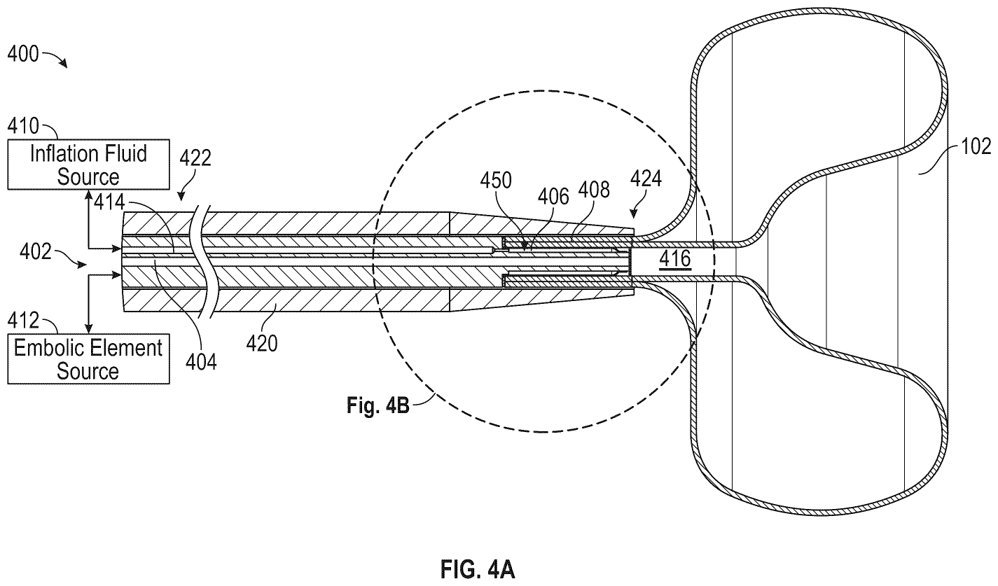

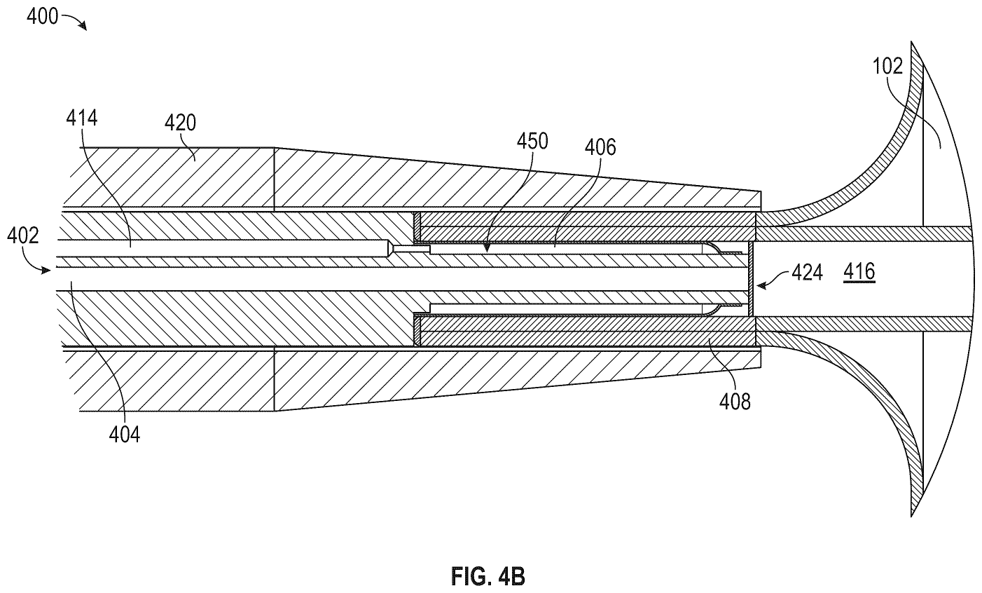

FIG. 1B is an enlarged view of the distal portion 100b of the treatment system 100. Referring to FIGS. 1A and 1B together, the occlusive member 102 may be detachably coupled to a distal portion of the elongated member 106. For example, the elongated member 106 may include a first coupler 112, and the occlusive member 102 may include a second coupler 114 configured to detachably couple with the first coupler 112. In some embodiments, the couplers 112, 114 can take the form of a detachment mechanism as described in more detail below with respect to FIGS. 4A-6B. The treatment system 100 may further comprise a conduit 116 extending from the handle 103 (for example, via port 110) distally to the distal portion 100b of the treatment system 100. The conduit 116 is configured to deliver the embolic element (and/or one or more precursors thereof) through one or more components of the delivery system (e.g., the first or second elongated shafts 109, 108, the elongated member 106, etc.) to a position at the exterior of the occlusive member 102. As such, the embolic element may be positioned between the occlusive member 102 and an inner wall of the aneurysm cavity, as described in greater detail below. In some embodiments, the elongated member 106 serves as the conduit 116.

According to some embodiments, the second elongated shaft 108 is generally constructed to track over a conventional guidewire in the cervical anatomy and into the cerebral vessels associated with the brain and may also be chosen according to several standard designs that are generally available. Accordingly, the second elongated shaft 108 can have a length that is at least 125 cm long, and more particularly may be between about 125 cm and about 175 cm long. In some embodiments, the second elongated shaft 108 may have an inner diameter of about 0.015 inches (0.0381 cm), 0.017 inches (0.043 cm), about 0.021 inches (0.053 cm), or about 0.027 inches (0.069 cm). Other designs and dimensions are contemplated.

The elongated member 106 can be movable within the first and/or second elongated shafts 109, 108 to position the occlusive member 102 at a desired location. The elongated member 106 can be sufficiently flexible to allow manipulation, e.g., advancement and/or retraction, of the occlusive member 102 through tortuous passages. Tortuous passages can include, for example, catheter lumens, microcatheter lumens, blood vessels, urinary tracts, biliary tracts, and airways. The elongated member 106 can be formed of any material and in any dimensions suitable for the task(s) for which the system is to be employed. In some embodiments, the elongated member 106 can comprise an elongated tubular member having a lumen therein, for example a conduit. In some embodiments, the elongated member 106 may comprise any other suitable form such as a solid metal wire, an elongated tubular shaft, or any combination thereof.

In some embodiments, the elongated member 106 can comprise stainless steel, nitinol, or other metal or alloy. In some embodiments, the elongated member 106 can be surrounded over some or all of its length by a coating, such as, for example, polytetrafluoroethylene. In some examples, the elongated member 106 can be a hypotube. The elongated member 106 may have a diameter that is generally constant along its length, or the elongated member 106 may have a diameter that tapers radially inwardly, along at least a portion of its length, as it extends in a distal direction.

An inflation fluid source 113 can be coupled to a proximal portion of the elongated shaft 106, which can take the form of an elongated tubular conduit. The inflation fluid source 113 can be configured to control the delivery of an inflation fluid (e.g., saline, air) along the tubular conduit (e.g., along an inflation lumen) to an expandable member that facilitates engagement of the elongated shaft 106 to the occlusive member 102 as described in more detail elsewhere herein. The inflation fluid source 113 can also facilitate removal of the inflation fluid from the expandable member. In various examples, the inflation fluid source 113 can include both a reservoir of the appropriate inflation fluid (e.g., saline, air, etc.) and a mechanism for introducing or removing the fluid such as a pump, syringe, etc.

### A. Selected Examples of Occlusive Members

FIG. 1C is a sectioned view of the occlusive member 102, shown in an expanded state and detached from the treatment system 100. Referring to FIGS. 1B and 1C, the occlusive member 102 may comprise an expandable element having a low-profile or constrained state while positioned within a catheter (such as the second elongated shaft 108) for delivery to the aneurysm and an expanded state in which the expandable element is configured to be positioned within an aneurysm (such as a cerebral aneurysm).

According to some embodiments, the occlusive member 102 may comprise a mesh 101 formed of a plurality of braided filaments that have been heat-set to assume a predetermined shape enclosing an interior volume 130 when the mesh 101 is in an expanded, unconstrained state. Example shapes include a globular shape, such as a sphere, a prolate spheroid, an oblate spheroid, and others. As depicted in FIG. 1C, the mesh 101 may have inner and outer layers 122, 124 that have proximal ends fixed relative to one another at the second coupler 114 and meet distally at a distal fold 128 surrounding an aperture 126. While the inner and outer layers 122, 124 are depicted spaced apart from one another along their lengths, the inner and outer layers 122, 124 may be in contact with one another along all or a portion of their lengths. For example, the inner layer 122 may press radially outwardly against the outer layer 124. In some embodiments, the occlusive member 102 may be formed of a single layer or mesh or braid.

In some embodiments, the inner and outer layers 122, 124 have their distal ends fixed relative to one another at a distal coupler and meet proximally at a proximal fold surrounding an aperture. In any case, in some embodiments the conduit 116 may be configured to be slidably positioned through some or all of the second coupler 114, the interior volume 130 of the expanded mesh 101, and the opening 126.

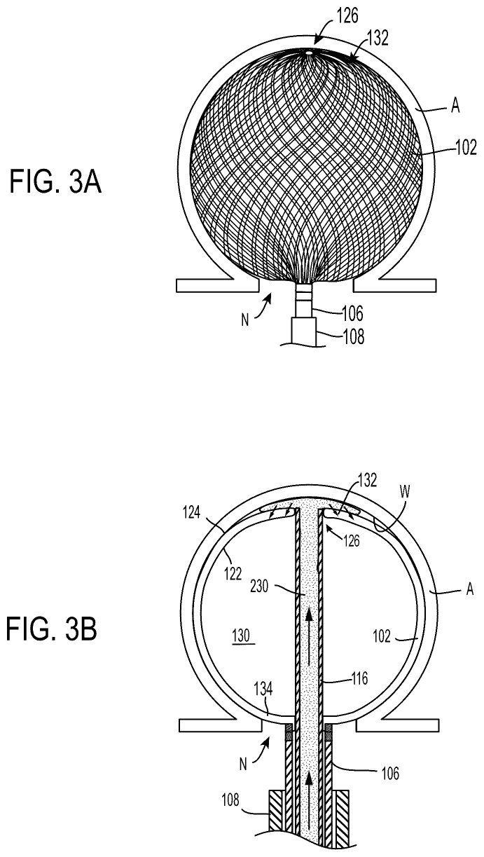

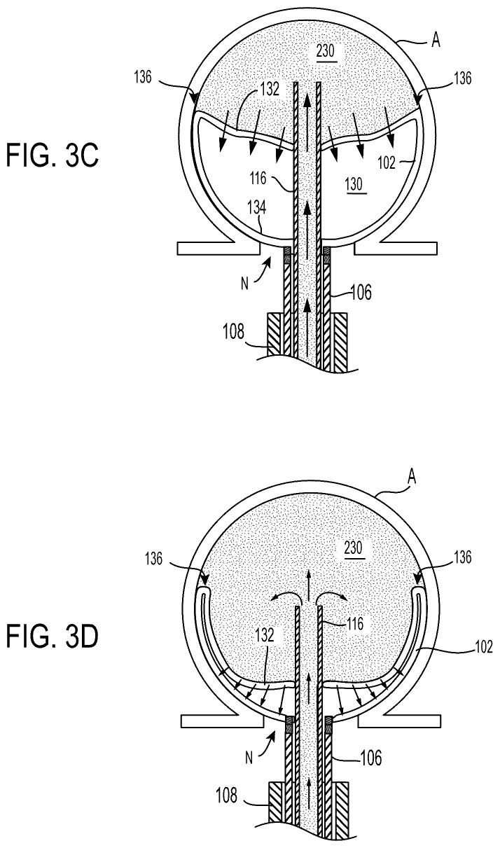

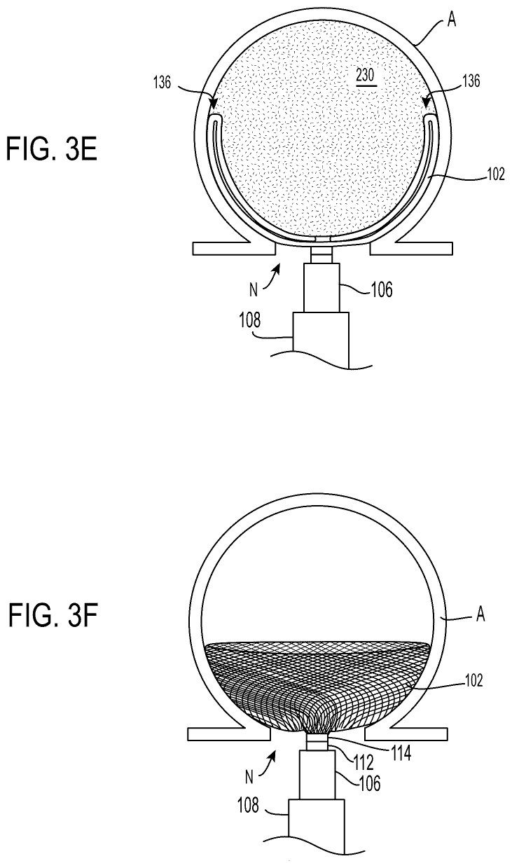

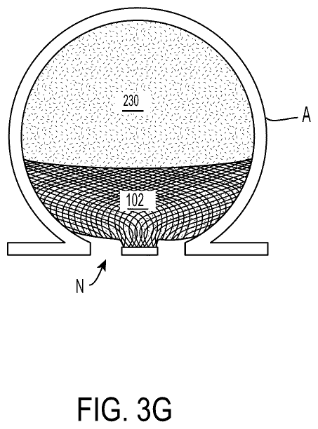

The inner and outer layers 122 and 124 may conform to one another at the distal portion (for example as shown in FIG. 1C) to form a curved distal surface. For example, at least at the distal portion of the occlusive member 102, the inner and outer layers 122 and 124 may extend distally and radially inwardly, towards the aperture 126. In some embodiments, the outer and/or inner layers 122 and 124 extend distally and radially outwardly from the second coupler 114, then extend distally and radially inwardly up to a distal terminus of the occlusive member 102 (e.g., the fold 128). The occlusive member 102 and/or layers thereof may be curved along its entire length, or may have one or more generally straight portions. In some embodiments, the curved surface transitions to a flat or substantially flat, distal-most surface that surrounds the aperture 126. In some embodiments, the curved surface transitions to a distal-most surface that surrounds the aperture 126 and has a radius of curvature that is greater than the average radius of curvature of the rest of the occlusive member 102. Having a flat or substantially flat distal surface, or a distal surface with a radius of curvature that is greater than the average radius of curvature of the rest of the occlusive member 102, may be beneficial for delivering the embolic element 230 in that it creates a small gap between the distal surface of the occlusive member 102 and the dome of the aneurysm A (see, for example, FIG. 3B). In some embodiments, the surface of the occlusive member 102 surrounding the aperture 126 is curved and/or has generally the same radius of curvature as the remainder of the occlusive member 102.

The inner layer 124 may have a shape that substantially conforms to the shape of the outer layer 124, or the inner and outer layers 122, 124 may have different shapes. For example, as shown in FIG. 1D, the inner layer 122 may have a diameter or cross-sectional dimension that is less than the outer layer 124. Such a configuration may be beneficial in that the embolic element 230 experiences less resistance, at least initially, when pushing the distal wall of the occlusion member 102 downwardly towards the neck (as described in greater detail below).

In any case, both the proximal portion and the distal portion of the mesh 101 can form generally closed surfaces. However, unlike at the proximal portion of the mesh 101, the portion of the filaments at or near the fold 128 at the distal portion of the mesh 101 can move relative to one another. As such, the distal portion of the mesh 101 has both the properties of a closed end and also some properties of an open end (like a traditional stent), such as some freedom of movement of the distal-most portions of the filaments and an opening through which the conduit 116, a guidewire, guidetube, or other elongated member may pass through.

The occlusive member 102 can have different shapes and sizes in an expanded, unconstrained state. For example, the occlusive member 102 may have a bullet shape, a barrel-shape, an egg shape, a dreidel shape, a bowl shape, a disc shape, a cylindrical or substantially cylindrical shape, a barrel shape, a chalice shape, etc.

### B. Selected Examples of Embolic Kits

The embolic kit 200 may include one or more precursors for creation of a liquid embolic. For example, the embolic kit 200 may include a first container 202 containing a first precursor material 203 (shown schematically), a second container 204 containing a second precursor material 205 (also shown schematically), and a mixing device 206 suitable for mixing the first and second precursor materials 203, 205. The mixing device 206 can include mixing syringes 208 (individually identified as mixing syringes 208a, 208b) and a coupler 210 extending between respective exit ports (not shown) of the mixing syringes 208. The mixing syringes 208a, 208b each include a plunger 212 and a barrel 214 in which the plunger 212 is slidably received.

The embolic kit 200 can further include an injection syringe 216 configured to receive a mixture of the first and second precursor materials 203, 205 and deliver the mixture to a proximal portion 100b of the treatment assembly 100. The injection syringe 216 can include a barrel 220, an exit port 222 at one end of the barrel 220, and a plunger 224 slidably received within the barrel 220 via an opposite end of the barrel 220. The handle 103 of the treatment system 100 may have a coupler configured to form a secure fluidic connection between the lumen and the exit port 222 of the injection syringe 216.

The first and second precursor materials 203, 205 can include a biopolymer and a chemical crosslinking agent, respectively. The chemical crosslinking agent can be selected to form covalent crosslinks between chains of the biopolymer. In some embodiments, the biopolymer of the first precursor material 203 includes chitosan or a derivative or analog thereof, and the chemical crosslinking agent of the second precursor material 205 includes genipin or a derivative or analog thereof.

Mixing the biopolymer of the first precursor material 203 and the chemical crosslinking agent of the second precursor material 205 can initiate chemical crosslinking of the biopolymer. After the first and second precursor materials 203, 205 are mixed, chemical crosslinking of the biopolymer occurs for enough time to allow the resulting embolic element 230 be delivered to the aneurysm before becoming too viscous to move through the lumen of the conduit 116. In addition, the period of time during which chemical crosslinking of the biopolymer occurs can be short enough to reach a target deployed viscosity within a reasonable time (e.g., in the range of 10-60 minutes; or at most 40 minutes, 30 minutes, 20 minutes, or 10 minutes) after delivery. The target deployed viscosity can be high enough to cause an agglomeration of the embolic element 230 to remain within the internal volume of the aneurysm without reinforcing the neck.

In some embodiments, the embolic kit 200 and/or embolic element 230 may be any embolic or occlusive device, such as one or more embolic coils, polymer hydrogel(s), polymer fibers, mesh devices, or combinations thereof. The embolic kit 200 may include one or more precursors that, once mixed together, form the embolic element 230 that remains within the aneurysm. In some embodiments, the embolic kit 200 may include the embolic element pre-mixed.

Additional details regarding suitable embolic elements may be found in U.S. patent application Ser. No. 15/299,929, filed Oct. 21, 2016, the disclosure of which is incorporated herein by reference in its entirety.

### II. SELECTED METHODS FOR TREATING ANEURYSMS

FIGS. 3A-3G depict an example method for treating an aneurysm A with the systems 10 of the present technology. To begin, a physician may intravascularly advance the second elongated shaft 108 towards an intracranial aneurysm (or other treatment location such as any of those described herein) with the occlusive member 102 in a low-profile state. A distal portion of the second elongated shaft 108 may be advanced through a neck N of the aneurysm A to locate a distal opening of the second elongated shaft 108 within an interior cavity of the aneurysm A. The elongated member 106 may be advanced distally relative to the second elongated shaft 108 to push the occlusive member 102 through the opening at the distal end of the second elongated shaft 108, thereby releasing the occlusive member 102 from the shaft 108 and allowing the occlusive member 102 to self-expand into a first expanded state. Releasing the occlusive member 102 from the shaft 108 and allowing the occlusive member 102 to self-expand into a first expanded state may alternatively, or additionally, include withdrawing shaft 108 relative to the elongated member 106.

FIG. 3A shows the occlusive member 102 in a first expanded state, positioned in an aneurysm cavity and still coupled to the elongated member 106. As shown in FIG. 3A, in the first expanded state, the occlusive member 102 may assume a predetermined shape that encloses an internal volume 130 (see FIG. 1C). In this first expanded state, the occlusive member 102 may generally conform to the shape of the aneurysm A. As illustrated in FIG. 3B with the occlusive member 102 and delivery system shown in cross-section, the conduit 116 may be advanced through the internal volume 130 of the occlusive member 102 such that a distal opening of the conduit 116 is at or distal to the aperture 126 at the distal portion of the occlusive member 102. The embolic element 230 may be delivered through the conduit 116 to a space between the occlusive member 102 and an inner surface of the aneurysm wall W. Although the illustrated example shows a separate conduit 116 extending through a lumen of the elongated member 106, in other embodiments the elongated member 106 may itself form the conduit 116, e.g., by extending through a proximal hub of the occlusive member 102 and through the internal volume 130.

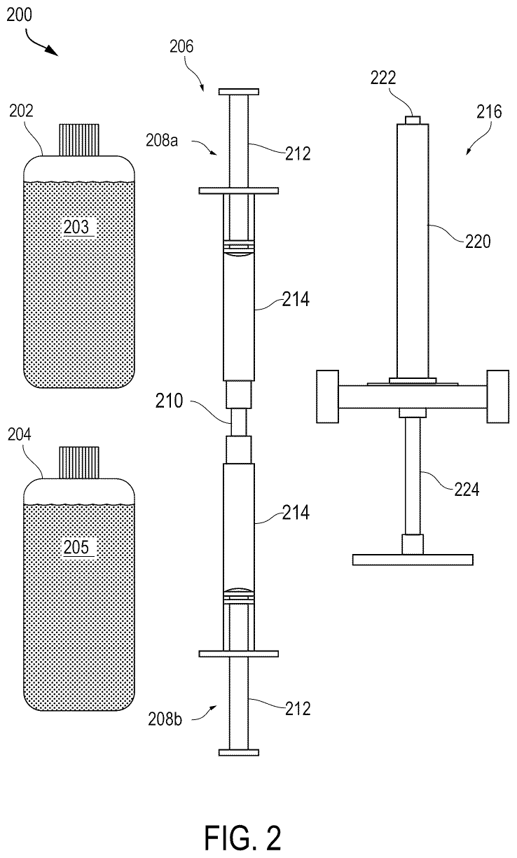

In some embodiments, the method includes mixing the first and second precursor materials 203, 205 (FIG. 2) to form the embolic element 230. Mixing of the first and second precursor materials 203, 205 may occur prior to introducing the embolic element 230 to the treatment system 100 and/or during delivery of the embolic element through the conduit 116 to the aneurysm. In a particular example, the first precursor material 203 is loaded into one of the barrels 214, the second precursor materials 205 is loaded into the other barrel 214, and the mixing syringes 208 are coupled via the coupler 210. To mix the first and second precursor materials 203, 205, the plungers 212 are alternately depressed, thereby causing the first and second precursor materials 203, 205 to move repeatedly from one barrel 214 to the other barrel 214. After suitably mixing the precursor materials, the resulting embolic element 230 can be loaded into the barrel 220 of the injection syringe 216. The injection syringe 216 may then be coupled to a proximal end of the conduit 116 to deliver the embolic element 230 through the conduit 116 and into the aneurysm A. As the embolic element 230 passes through the lumen of the conduit 116, chemical crosslinking of the biopolymer can continue to occur.

Still with reference to FIG. 3B, as the embolic element 230 is delivered between the dome of the aneurysm A and the distal portion 132 of the wall of the occlusive member 102, pressure builds between the aneurysm wall W and the occlusive member 102. As shown in the progression of FIGS. 3B-3D, when the forces on the occlusive member 102 reach a threshold level, the embolic element 230 pushes the distal wall 132 downwardly towards the neck N of the aneurysm A. The embolic element 230 exerts a substantially uniform pressure across the distal surface of the occlusive member 102 that collapses the occlusive member 102 inwardly on itself such that the rounded distal wall 132 transitions from concave towards the neck N of the aneurysm A to convex towards the neck N. The pressure and inversion of the distal portion of the wall 132 creates an annular fold 136 that defines the distal-most edge of the occlusive member 102. As the occlusive member 102 continues to invert, the position of the fold 136 moves towards the neck N, which continues until a distal-most half of the occlusive member 102 has inverted. In some embodiments, the occlusive member 102 may include one or more portions configured to preferentially flex or bend such that the occlusive member 102 folds at a desired longitude. Moreover, as the occlusive member 102 collapses, a distance between the wall at the distal portion 132 and the wall at the proximal portion decreases, and thus the internal volume 130 of the occlusive member 102 also decreases. As the occlusive member 102 collapses, the conduit 116 may be held stationary, advanced distally, and/or retracted proximally.

During and after delivery of the embolic element 230, none or substantially none of the embolic element 230 migrates through the pores of the occlusive member 102 and into the internal volume 130. Said another way, all or substantially all of the embolic element 230 remains at the exterior surface or outside of the occlusive member 102. Compression of the occlusive member with the embolic element 230 provides a real-time “leveling” or “aneurysm-filling indicator” to the physician under single plane imaging methods (such as fluoroscopy) so that the physician can confirm at what point the volume of the aneurysm is completely filled. It is beneficial to fill as much space in the aneurysm as possible, as leaving voids within the aneurysm sac may cause delayed healing and increased risk of aneurysm recanalization and/or rupture. While the scaffolding provided by the occlusive member 102 across the neck helps thrombosis of blood in any gaps and healing at the neck, the substantial filling of the cavity prevents rupture acutely and does not rely on the neck scaffold (i.e., the occlusive member 102). Confirmation of complete or substantially complete aneurysm filling under single plane imaging cannot be provided by conventional devices.

Once delivery of the embolic element 230 is complete, the conduit 116 may be withdrawn. In some embodiments, the embolic element 230 may fill greater than 40% of the aneurysm sac volume. In some embodiments, the embolic element 230 may fill greater than 50% of the aneurysm sac volume. In some embodiments, the embolic element 230 may fill greater than 60% of the aneurysm sac volume. In some embodiments, the embolic element may fill greater than 65%, 70%, 75%, 80%, 85%, or 90% of the aneurysm sac volume.

FIG. 3E shows a second expanded state of the occlusive member 102, shown in cross-section, with the embolic element 230 occupying the remaining volume of the aneurysm A. FIG. 3F shows the occlusive member 102 in full with the embolic element 230 removed so the second shape of the occlusive member 102 is visible. As shown, the embolic element 230 may be delivered until the occlusive member 102 is fully collapsed such that the occlusive member 102 has substantially no internal volume.

In the second expanded state, the occlusive member 102 may form a bowl shape that extends across the neck of the aneurysm A. The wall of the occlusive member 102 at the distal portion may now be positioned in contact with or immediately adjacent the wall of the occlusive member 102 at the proximal portion. The distal wall 132 may be in contact with the proximal wall 134 along all or substantially all of its length. In some embodiments, the distal wall 132 may be in contact with the proximal wall 134 along only a portion of its length, while the remainder of the length of the distal wall 132 is in close proximity—but not in contact with—the proximal wall 134.

Collapse of the occlusive member 102 onto itself, towards the neck N of the aneurysm, may be especially beneficial as it doubles the number of layers across the neck and thus increases occlusion at the neck N. For example, the distal wall 132 collapsing or inverting onto the proximal wall 134 may decrease the porosity of the occlusive member 102 at the neck N. In those embodiments where the occlusive member 102 is a mesh or braided device such that the distal wall 132 has a first porosity and the proximal wall 134 has a second porosity, deformation of the distal wall 132 onto or into close proximity within the proximal wall 134 decreases the effective porosity of the occlusive member 102 over the neck N. The resulting multi-layer structure thus has a lower porosity than the individual first and second porosities. Moreover, the embolic element 230 along the distal wall 132 provides additional occlusion. In some embodiments, the embolic element 230 completely or substantially completely occludes the pores of the adjacent layer or wall of the occlusion member 102 such that blood cannot flow past the embolic element 230 into the aneurysm cavity. It is desirable to occlude as much of the aneurysm as possible, as leaving voids of gaps can allow blood to flow in and/or pool, which may continue to stretch out the walls of aneurysm A. Dilation of the aneurysm A can lead to recanalization and/or herniation of the occlusive member 102 and/or embolic element 230 into the parent vessel and/or may cause the aneurysm A to rupture. Both conditions can be fatal to the patient.

In those embodiments where the wall of the occlusive member 102 comprises an inner and outer layer, the deformed or second shape of the occlusive member 102 forms four layers over the neck N of the aneurysm A. In those embodiments where the wall of the occlusive member 102 comprises a single layer, the deformed or second shape of the occlusive member 102 forms two layers over the neck N of the aneurysm A As previously mentioned, the neck coverage provided by the doubled layers provides additional surface area for endothelial cell growth, decreases the porosity of the occlusive member 102 at the neck N (as compared to two layers or one layer), and prevents herniation of the embolic element 230 into the parent vessel. During and after delivery, the embolic element 230 exerts a substantially uniform pressure on the occlusive member 102 towards the neck N of the aneurysm A, thereby pressing the portions of the occlusive member 102 positioned adjacent the neck against the inner surface of the aneurysm wall such that the occlusive member 102 forms a complete and stable seal at the neck N.

As shown in FIG. 3G, the first coupler 112 may be detached from the second coupler 114 and the elongated member 106 and second elongated shaft 108 may be withdrawn, thereby leaving the occlusive member 102 and embolic element 230 implanted within the aneurysm A. For example, the occlusive member 102 may be detached from the elongated member 106 using any of the detachment mechanisms described in more detail below. In some examples, at least a distal portion of the conduit 116 may remain in place following detachment (e.g., by actuation of an expandable member such as a balloon) of the elongated member 106 and/or conduit 116.

### III. EXAMPLE SYSTEMS WITH EXPANDABLE MEMBER DETACHMENT MECHANISMS

As noted previously, following delivery of the occlusive member 102 and/or an embolic element 230 to a treatment site, the occlusive member 102 can be released from the conduit using a detachment mechanism. In various examples described herein, the detachment mechanism can include an expandable member coupled to the conduit that can transition between (1) an engaged (e.g., attached) configuration in which an expandable member is radially expanded to frictionally and/or mechanically engage a proximal hub of the occlusive member 102, and (2) a disengaged (e.g., detached) configuration in which the expandable member is radially collapsed such that it no longer frictionally and/or mechanically engages the proximal hub of the occlusive member 102. Once disengaged, the conduit can be removed from the body while the occlusive member 102 and the embolic element 230 remain in place at the treatment site.

FIG. 4A illustrates a side cross-sectional view of a treatment system 400, and FIG. 4B illustrates an enlarged detail view of portion 4B labeled in FIG. 4A. The treatment system 400 can include some or all of the features of the treatment system 100 described elsewhere herein. With reference to FIGS. 4A and 4B together, the treatment system 400 includes a conduit 402 releasably coupled to an occlusive member 102 via an expandable member 406, which is coupled to an engagement portion 450 of the conduit 402. In operation, the expandable member 406 can be manipulated to engage and disengage the occlusive member 102 from the conduit engagement portion 450.

With reference to FIG. 4A, the conduit 402 can take the form of an elongated tubular member such as a hypotube, catheter, etc., and can be slidably disposed within a surrounding tubular member 420 (e.g., a microcatheter). The lumen of the surrounding tubular member 420 can include a lubricious coating to allow for sliding of the conduit 402 therethrough. The conduit 402 defines a conduit lumen 404 extending from an extracorporeally located proximal end portion 422 to a distal end portion 424, which is releasably coupled to the occlusive member 102. In at least some implementations, the conduit lumen 404 is omitted, in which case the conduit 402 serves as a core member for delivering an implant such as occlusive member 102 but does not facilitate delivery of an embolic element through an internal lumen.

As seen in FIG. 4A, the conduit engagement portion 450 can include a distal portion of the conduit 402 that is disposed within a proximal hub 408 of the occlusive member 102. The engagement portion 450 may include a stepped-down outer diameter, such that the outermost cross-sectional dimension of the conduit 402 is smaller along the engagement portion 450 than along more proximal regions of the conduit 402. The outermost cross-sectional dimension of the engagement portion 450 can be smaller than an inner dimension of the proximal hub 408, such that there is a radial gap between the radially outer surface of the engagement portion 450 of the conduit and the radially inner surface of the proximal hub 408. The expandable member 406 can be coupled to the radially outer surface of the engagement portion 450 of the conduit 402 and be positioned so as to be disposed within this radial gap. In this configuration, the expandable member 406 underlies at least a portion of the proximal hub 408.

In various embodiments, the conduit 402 can have a length sufficient to permit the occlusive member 102 to be positioned at an intravascular treatment site (e.g., within an aneurysm sac) while a proximal end of the conduit 402 extends outside the patient's body. For example, the conduit 402 can have a length of greater than about 50 inches, 60 inches, 70 inches, or 80 inches. The conduit 402 can have an outer diameter suitable to permit the conduit 402 to be slidably advanced through a surrounding tubular member 420. For example, the conduit 402 can have an outer diameter of less than about 0.027 inches, less than about 0.021 inches, or less than about 0.017 inches. In some embodiments, the conduit 402 can have a wall thickness of between about 0.0005 inches and about 0.0015 inches, or about 0.001 inches. In some embodiments, the conduit 402 has a stepped down outer diameter along the engagement portion 450 which can be releasably disposed within the proximal hub 408 of the occlusive member 102. For example, conduit 402 can have an outer diameter of about 0.016 inches proximal to the engagement portion 450, and an outer diameter of about 0.014 inches along the engagement portion 450. In some embodiments, the conduit 402 need not have such a stepped-down outer diameter. For example, the conduit 402 can have an outer diameter and/or an inner diameter that is substantially constant along its length, or that tapers gradually along some or all of its length.

The conduit 402 defines a conduit lumen 404 along its length. The conduit lumen 404 is distally coupled to the embolic member 102. In operation, an embolic element (e.g., an embolic liquid or gel, coils, etc.) from an extracorporeal embolic element source 412 (e.g., a syringe, pump, reservoir, etc.) can be introduced into a proximal portion of the conduit lumen 404. The embolic element is then delivered distally through the conduit lumen 404 until it passes from the distal end of the conduit lumen 404 to be delivered to the treatment site as described above with respect to FIGS. 3A-3G. The conduit lumen 404 can be coated with a lubricious material or lining to facilitate advancement of embolic element(s) therethrough.

In some embodiments, the conduit 402 is dimensioned such that the distal opening is disposed adjacent to, completely distal of, or at least partially distal of the occlusive member 102 while the occlusive member 102 is in the unexpanded state. In the illustrated example, the distal end of the conduit lumen 404 abuts an implant lumen 416 defined by the occlusive member 102. For example, the occlusive member 102 can be formed as a tubular structure in which both ends of the tube are affixed to the proximal hub 408, thereby defining an implant lumen 416 extending distally way from the proximal hub 408. In operation, an embolic element delivered through the conduit lumen 404 passes through the implant lumen 416 to a delivery site distal to at least a portion of the occlusive member 102.

In some embodiments, the hub 408 includes an inner band that circumferentially surrounds the engagement portion 450 of the conduit 402, and an outer band that surrounds the inner band, such that proximal portions of the layers of the occlusive member 102 are grasped between the inner and outer bands of the hub 408. Such bands can be made of any suitable material, for example polymeric or metallic, and optionally may be radiopaque to facilitate visualization of the system 400 as it advanced through the vasculature. The bands can be crimped, with or without an adhesive or weld, to secure them in place. In some examples, hub 408 can have an inner diameter of about 0.020 inches and an outer diameter of about 0.023 inches.

In some implementations, the hub 408 can take the form of a “crimpless” hub in which proximal ends of the occlusive member 102 are secured using a cured flowable material, omitting the need for any bands or other mechanical constraints. This approach can reduce the outer diameter of the hub 408, since the cured material may add only a nominal amount to the overall thickness/diameter of the bundled filaments, thus providing 25-50% more space within the delivery catheter (relative to bands) that can be used to enlarge the fluid channel running through the secured filament ends. Additional details regarding such a hub configuration can be found in commonly owned U.S. application Ser. No. 17/816,380, filed Jul. 29, 2022, titled “Devices, Systems, and Methods for Treating Aneurysms,” which is hereby incorporated by reference in its entirety.

As illustrated in FIG. 4A, the conduit 402 also defines an inflation lumen 414 therein. The inflation lumen 414 can extend alongside the conduit lumen 404 along at least a portion of the conduit 402. In alternative implementations, the inflation lumen 414 can be provided in a separate member (e.g., a separate inflation tube) that runs adjacent to the conduit 402. The inflation lumen 414 can be proximally coupled to an inflation fluid source 410 (e.g., an extracorporcally located syringe, pump, reservoir, etc.) configured to supply a suitable inflation fluid (e.g., saline, air, etc.) and/or to remove the inflation fluid from the inflation lumen 414. In some embodiments, the inflation fluid can be saline of physiologic composition such that the inflation fluid causes no harm to the patient upon any leakage from the inflation lumen 414. In other embodiments, the inflation fluid can be other fluids (e.g., water, mixtures, suspensions, solutions, vapors, gases). The inflation lumen 414 is distally coupled to the expandable member 406 as described in more detail below. In operation, inflation fluid delivered or removed via the inflation lumen 414 causes the expandable member 406 to expand or collapse, respectively, thereby engaging or disengaging the occlusive member 102 with respect to the conduit 402.

When in the engaged configuration, the expandable member 406 can expand to a first dimension substantially equivalent to the inner diameter of the proximal hub 408 (or greater than the inner diameter of the proximal hub 408 when not constrained by the presence of the proximal hub 408). When in the disengaged configuration, the expandable member 406 can collapse to a second dimension smaller than the inner diameter of the proximal hub 408 which is required to releasably couple the conduit 402 from the occlusive member 102.

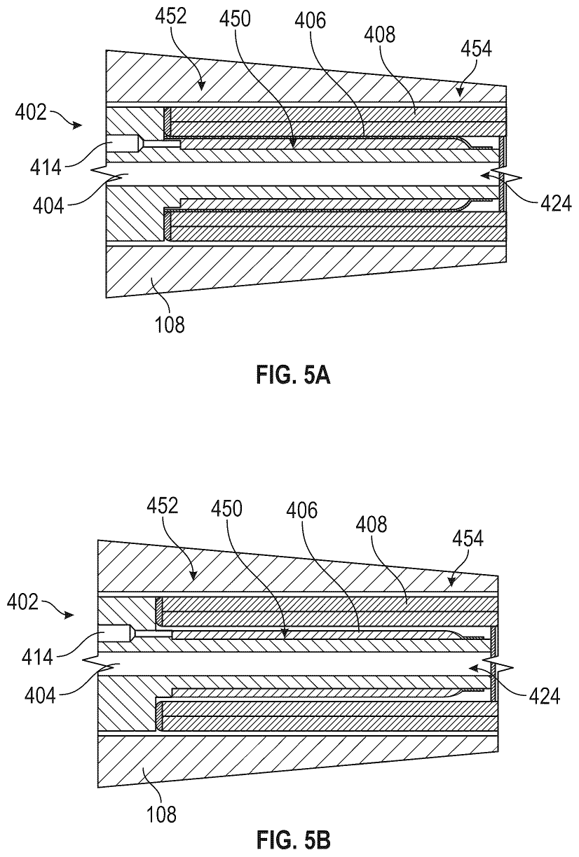

FIG. 5A illustrates an enlarged cross-sectional side view of a portion of the treatment system 400 in the engaged configuration. FIG. 5B illustrates an enlarged cross-sectional side view of the portion of the treatment system 400 in the disengaged configuration. With reference to FIGS. 5A-5B together, the expandable member 406 is coupled to (e.g., carried by or attached to) a radially outer surface of the conduit 402 along the engagement portion 450. The expandable member 406 can take the form of a balloon or other inflatable member, and may be in fluid communication with the inflation lumen 414 such that fluid (e.g., saline, air) delivered through the inflation lumen 414 enters into the expandable member 406 to expand (e.g., inflate) the expandable member 406. Similarly, fluid can be drawn out of the expandable member 406 via the inflation lumen 414, thereby collapsing (e.g., deflating) the expandable member 406. In the illustrated example, the expandable member takes the form of an annular balloon extending from a proximal end portion 452 to a distal end portion 454 along substantially all of the engagement portion 450 of the conduit 402. In some embodiments, the expandable member 406 can extend the length of the stepped down region of the engagement portion 450 of the conduit 402. Alternatively, the expandable member 406 can be any suitable length to achieve the engaged configuration of the occlusive member 406 and the conduit 402.

As best seen in FIG. 5A, in the engaged configuration, the expandable member 406 is radially expanded such that a radially outer surface of the expandable member 406 contacts the radially inner surface of the proximal hub 408 of the occlusive member 102. This contact creates frictional engagement, such that movement of the conduit 402 (and thereby movement of the expandable member 406) causes corresponding movement of the occlusive member 102. In some implementations, engagement between the expandable member 406 and the inner surface of the proximal hub 408 can be increased by surface treatment or surface features provided on one or both of the contact surfaces. For instance, the radially outer surface of the expandable member 406 and/or the radially inner surface of the proximal hub 408 can be provided with surface treatment to increase friction (e.g., roughening one or both surfaces, such as providing a sandpaper-like finish to one or both surfaces). Additionally or alternatively, one or both surfaces can be provided with protrusions, barbs, bumps, ridges, etc. One or both surfaces can likewise be provided with grooves, recesses, detents, etc. In some instances, corresponding male and female features on the opposing contact surfaces can be configured to mate such that the proximal hub 408 and the expandable member 406 mechanically interlock with one another.

FIG. 5B illustrates a disengaged configuration, in which the expandable member 406 has been collapsed to a low-profile (e.g., deflated) configuration. In this state, the radially outermost dimension of the expandable member 406 has been reduced relative to the engaged state shown in FIG. 5A, such that the expandable member 406 exerts little or no radially outward force on the proximal hub. For instance, the radially outward force provided by the expandable member 406 against the proximal hub 408 can be lower in the disengaged configuration (FIG. 5B) than in the engaged configuration (FIG. 5A). In the disengaged configuration, due to the reduced or eliminated radially outward force of the expandable member 406, frictional engagement between the expandable member 406 and the proximal hub 408 is reduced or eliminated. As such, the engagement portion 450 of the conduit 402 can be freely slidably removed from the proximal hub 408 of the occlusive member 102, thereby releasing the occlusive member 102 from the conduit 402.

In various examples, the engaged configuration shown in FIG. 5A in which the expandable member 406 assumes its expanded state can be achieved by supplying an inflation fluid (e.g., saline, air) at sufficient pressure along the inflation lumen 414 that the expandable member 406 maintains its expanded state and exerts a radially outward force on the proximal hub 408. Conversely, the disengaged configuration shown in FIG. 5B can be achieved by removing an inflation fluid (e.g., saline, air) from the expandable member 406, for instance by supplying negative pressure along the inflation lumen 414, thereby causing the expandable member 406 to collapse and assume a low-profile configuration.

In some implementations, the expandable member 406 can be self-expanding, for instance incorporating a self-expanding stent or other structure that provides a radially outer force irrespective of the inflation fluid. In such implementations, the expandable member 406 is biased towards the expanded state, and a negative pressure supplied via the inflation lumen 414 can be provided to overcome this radially outward force to move the expandable member 406 into the collapsed configuration shown in FIG. 5B. In some implementations, the expandable member 406 can be biased (e.g., including a stent or other structure that is shape set) towards the collapsed configuration, such that when no external force is applied the expandable member 406 assumes the collapsed configuration. In such instances, positive pressure supplied via the inflation lumen 414 can be used to overcome this bias and force the expandable member 406 into the expanded state.

In various examples, the expandable member 406 can be annular and can surround the conduit 402 circumferentially along the engagement portion 450. In other embodiments, the expandable member 406 may only surround a portion of the conduit 402. Furthermore, in other embodiments, there may be multiple expandable members 406 coupled to the conduit 402 and one or more inflation lumens 414 of the conduit 402.

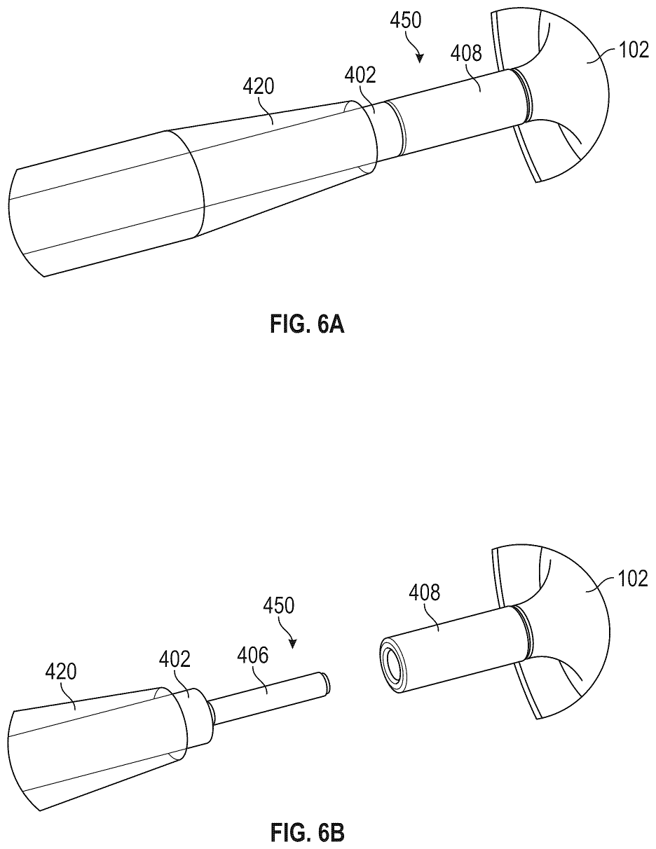

FIG. 6A illustrates a perspective view of a portion of the treatment system 400 in the engaged configuration, and FIG. 6B illustrates a perspective view of the treatment system 400 in the disengaged configuration. During advancement of the treatment system 400 through vasculature and/or optional injection of an embolic element into a cavity of the treatment location (e.g., a position distal to at least a portion of a distal face of the occlusive member 102), the expandable member is coupled to the conduit 402 as shown in FIG. 6A. The inflation fluid provides a radially outward force on the expandable member 406, which exerts a radially outward force on the radially inner surface of the proximal hub 408 to prevent axial movement of the proximal hub 408 with respect to the engagement portion 450 of the conduit 402.

As seen in FIG. 6B, following release of the occlusive member 102, the remainder of the treatment system 400 can be proximally retracted while the occlusive member 102 remains in place. To release the occlusive member 102, the expandable member 406 is collapsed to a low-profile configuration (e.g., by withdrawing fluid via the inflation lumen), thereby causing the expandable member 406 to disengage the proximal hub 408. At this point, the conduit 402 is in a disengaged configuration with the occlusive member 102 and can be slidably retracted proximally with respect to the proximal hub 408. Optionally, the surrounding tubular member 420 can be proximally retracted as well.

### IV. CONCLUSION

Although many of the embodiments are described above with respect to systems and methods related to treatment of hemorrhagic stroke, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-6B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Unless otherwise indicated, all numbers expressing dimensions, percentages, or other numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The disclosed technology is illustrated, for example, according to various examples described below. Various examples of examples of the disclosed technology are described as numbered examples (1, 2, 3, etc.) for convenience. These are provided as examples and do not limit the disclosed technology. It is noted that any of the dependent examples may be combined in any combination, and placed into a respective independent example. The other examples can be presented in a similar manner.

Example 1. A treatment system comprising: an occlusive implant comprising a proximal hub having a radially inner surface defining a hub lumen, the implant configured to be positioned at an intrasaccular treatment site; a conduit comprising an engagement portion configured to be releasably disposed within the hub lumen, the conduit defining a conduit lumen extending along its length and configured to pass an embolic element therethrough; and an expandable member coupled to a radially outer surface of the conduit engagement portion and configured to transition between an engaged configuration and a disengaged configuration, wherein: in the engaged configuration, the expandable member is expanded such that the expandable member exerts a radially outward force on the radially inner surface of the proximal hub to prevent axial movement of the proximal hub with respect to the conduit engagement portion; and in the disengaged configuration, the expandable member is collapsed such that the conduit engagement portion can be slidably retracted with respect to the proximal hub.

Example 2. The treatment system of any one of the preceding Examples, wherein the expandable member comprises a balloon, and wherein expanding the expandable member comprises inflating the balloon, and wherein collapsing the expandable member comprises deflating the balloon.

Example 3. The treatment system of any one of the preceding Examples, further comprising an inflation lumen running alongside the conduit lumen and in fluid communication with the balloon.

Example 4. The treatment system of any one of the preceding Examples, wherein the expandable member circumferentially surrounds the conduit engagement portion.

Example 5. The treatment system of any one of the preceding Examples, wherein the conduit engagement portion has a smaller outermost cross-sectional dimension than a proximal portion of the conduit.

Example 6. The treatment system of any one of the preceding Examples, wherein the conduit comprises a distal end portion extending distal to the conduit engagement portion.

Example 7. The treatment system of any one of the preceding Examples, wherein the implant defines an implant lumen in fluid communication with the conduit lumen when in the engaged state, the implant lumen configured to pass an embolic material therethrough to a position distal to at least a portion of a distal face of the implant.

Example 8. The treatment system of any one of the preceding Examples, wherein the occlusive implant comprises a self-expandable braided implant.

Example 9. The treatment system of any one of the preceding Examples, wherein in the engaged configuration, a radially outer surface of the expandable member mechanically interlocks with the radially inner surface of the proximal hub.

Example 10. A treatment system comprising: a detachable medical device comprising a proximal hub having a radially inner surface defining a hub lumen; a core member comprising an engagement portion configured to be releasably disposed within the hub lumen; and an expandable member coupled to a radially outer surface of the core member engagement portion and configured to transition between an engaged configuration and a disengaged configuration, wherein: in the engaged configuration, the expandable member is expanded such that the expandable member exerts a radially outward force on the radially inner surface of the proximal hub to prevent axial movement of the proximal hub with respect to the core member engagement portion; and in the disengaged configuration, the expandable member is collapsed such that the core member engagement portion can be slidably retracted with respect to the proximal hub.

Example 11. The treatment system of any one of the preceding Examples, wherein the expandable member comprises a balloon, and wherein expanding the expandable member comprises inflating the balloon, and wherein collapsing the expandable member comprises deflating the balloon.

Example 12. The treatment system of any one of the preceding Examples, further comprising an inflation lumen running along the core member and in fluid communication with the balloon.

Example 13. The treatment system of any one of the preceding Examples, wherein the core member comprises a conduit defining a conduit lumen configured to pass an embolic element therethrough.

Example 14. The treatment system of any one of the preceding Examples, wherein the occlusive implant comprises a self-expandable braided implant.

Example 15. The treatment system of any one of the preceding Examples, wherein in the engaged configuration, a radially outer surface of the expandable member mechanically interlocks with the radially inner surface of the proximal hub.

Example 16. A method comprising: disposing an occlusive implant at a treatment site, the implant having a proximal hub releasably coupled to a conduit engagement portion via an expandable member in an expanded state; expanding the occlusive implant at the treatment site; collapsing the expandable member to disengage the conduit from the proximal hub; and proximally retracting conduit and the expandable member while the occlusive implant remains at the treatment site.

Example 17. The method of any one of the preceding Examples, wherein the treatment site comprises an aneurysm sac.

Example 18. The method of any one of the preceding Examples, further comprising, after expanding the occlusive implant and before collapsing the expandable member, delivering an embolic element through the conduit to the treatment site.

Example 19. The method of any one of the preceding Examples, wherein the expandable member comprises a balloon disposed over an exterior surface of the conduit engagement portion, and wherein collapsing the expandable member comprises deflating the balloon.

Example 20. The method of any one of the preceding Examples, wherein, before collapsing the expandable member, the expandable member exerts a radially outward force on a radially inner surface of the implant proximal hub.

1. A treatment system comprising:
an occlusive implant comprising a proximal hub having a radially inner surface defining a hub lumen, the implant configured to be positioned at an intrasaccular treatment site;
a conduit comprising an engagement portion configured to be releasably disposed within the hub lumen, the conduit defining a conduit lumen extending along its length and configured to pass an embolic element therethrough; and
an expandable member coupled to a radially outer surface of the conduit engagement portion and configured to transition between an engaged configuration and a disengaged configuration, wherein:
in the engaged configuration, the expandable member is expanded such that the expandable member exerts a radially outward force on the radially inner surface of the proximal hub to prevent axial movement of the proximal hub with respect to the conduit engagement portion; and
in the disengaged configuration, the expandable member is collapsed such that the conduit engagement portion can be slidably retracted with respect to the proximal hub.

2. The treatment system of claim 1, wherein the expandable member comprises a balloon, and wherein expanding the expandable member comprises inflating the balloon, and wherein collapsing the expandable member comprises deflating the balloon.

3. The treatment system of claim 2, further comprising an inflation lumen running alongside the conduit lumen and in fluid communication with the balloon.

4. The treatment system of claim 1, wherein the expandable member circumferentially surrounds the conduit engagement portion.

5. The treatment system of claim 1, wherein the conduit engagement portion has a smaller outermost cross-sectional dimension than a proximal portion of the conduit.

6. The treatment system of claim 1, wherein the conduit comprises a distal end portion extending distal to the conduit engagement portion.

7. The treatment system of claim 1, wherein the implant defines an implant lumen in fluid communication with the conduit lumen when in the engaged state, the implant lumen configured to pass an embolic material therethrough to a position distal to at least a portion of a distal face of the implant.

8. The treatment system of claim 1, wherein the occlusive implant comprises a self-expandable braided implant.

9. The treatment system of claim 1, wherein in the engaged configuration, a radially outer surface of the expandable member mechanically interlocks with the radially inner surface of the proximal hub.

10. A treatment system comprising:
a detachable medical device comprising a proximal hub having a radially inner surface defining a hub lumen;
a core member comprising an engagement portion configured to be releasably disposed within the hub lumen; and
an expandable member coupled to a radially outer surface of the core member engagement portion and configured to transition between an engaged configuration and a disengaged configuration, wherein:
in the engaged configuration, the expandable member is expanded such that the expandable member exerts a radially outward force on the radially inner surface of the proximal hub to prevent axial movement of the proximal hub with respect to the core member engagement portion; and
in the disengaged configuration, the expandable member is collapsed such that the core member engagement portion can be slidably retracted with respect to the proximal hub.

11. The treatment system of claim 10, wherein the expandable member comprises a balloon, and wherein expanding the expandable member comprises inflating the balloon, and wherein collapsing the expandable member comprises deflating the balloon.

12. The treatment system of claim 11, further comprising an inflation lumen running along the core member and in fluid communication with the balloon.

13. The treatment system of claim 10, wherein the core member comprises a conduit defining a conduit lumen configured to pass an embolic element therethrough.

14. The treatment system of claim 10, wherein the occlusive implant comprises a self-expandable braided implant.

15. The treatment system of claim 10, wherein in the engaged configuration, a radially outer surface of the expandable member mechanically interlocks with the radially inner surface of the proximal hub.

16. A method comprising:
disposing an occlusive implant at a treatment site, the implant having a proximal hub releasably coupled to a conduit engagement portion via an expandable member in an expanded state;
expanding the occlusive implant at the treatment site;
collapsing the expandable member to disengage the conduit from the proximal hub; and
proximally retracting conduit and the expandable member while the occlusive implant remains at the treatment site.

17. The method of claim 16, wherein the treatment site comprises an aneurysm sac.

18. The method of claim 16, further comprising, after expanding the occlusive implant and before collapsing the expandable member, delivering an embolic element through the conduit to the treatment site.

19. The method of claim 16, wherein the expandable member comprises a balloon disposed over an exterior surface of the conduit engagement portion, and wherein collapsing the expandable member comprises deflating the balloon.

20. The method of claim 16, wherein, before collapsing the expandable member, the expandable member exerts a radially outward force on a radially inner surface of the implant proximal hub.
